(19) United States
(12) Patent Application Publication
(10) Pub. No.: US 2007/0082046 A1
(43) Pub. Date: Apr. 12, 2007

(54) Enteric valproic acid

(75) Inventors: Nachiappan Chidambaram, High Point, NC (US); Aqeel Fatmi, Greensboro, NC (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Banner Pharmacaps, Inc.

(21) Appl. No.: 11/247,389

(22) Filed: Oct. 11, 2005

(51) Int. Cl. A61K 9/64; A61K 31/19

(52) U.S. Cl. 424/456; 514/557

(57) ABSTRACT

An enteric valproic acid soft gelatin capsule, in which the enteric polymer is a component of the capsule shell rather than a coating, has been developed. The fill material comprises valproic acid or divalproex sodium and, optionally, one or more pharmaceutically acceptable excipients such as corn oil. The capsule shell is prepared from a mass comprising a film-forming polymer, an acid insoluble polymer, an aqueous solvent, and optionally a plasticizer. Suitable film-forming polymers include gelatin. Suitable acid-insoluble polymers include acrylic-acid/methacrylic acid copolymers. The acid-insoluble polymer is present in an amount from about 8% to about 20% by weight of the wet gel mass. The weight ratio of acid-insoluble polymer to film-forming polymer is from about 25% to about 50%. The aqueous solvent is water or an aqueous solution of alkalis such as ammonia or diethylene amine or hydroalcoholic solutions of the same. Suitable plasticizers include glycerin and triethylcitrate. The enteric soft gelatin capsule does not require an enteric coating and thus is not susceptible to the processing problems associated with enteric coated dosage forms. Enteric valproic acid soft gelatin capsules may be smaller in size and thus easier to swallow than currently available enteric coated tablets due to the presence of fewer ingredients, as well as smaller amounts of ingredients, in the capsule shell.

### FIELD OF THE INVENTION

This invention is in the field of pharmaceutical compositions, specifically an enteric valproic acid gelatin capsule formulation.

### BACKGROUND OF THE INVENTION

Valproic Acid, or 2-propylpentanoic acid, and its salts and derivatives are used to treat absence seizures, complex partial seizures, mania, migraine headache prophylaxis, and behavior dyscontrol. Once in the body, valproic acid and its salts and derivatives are converted to valproate ion, which is responsible for the therapeutic effect. Valproic acid and its salts and derivatives are also known to cause significant side effects including gastrointestinal discomfort (nausea, indigestion, vomiting, diarrhea, and abdominal pain) which can decrease patient compliance.

Valproic acid and sodium valproate are difficult to formulate into solid oral dosage forms. Sodium valproate is extremely hygroscopic, often liquifying rapidly under ambient conditions. Valproic acid is an oily liquid at room temperature and thus not suitable for manufacturing solid dosage forms, e.g. tablets for oral administration.

Efforts have been made to address the problems associated with formulating valproic acid and sodium valproate into solid oral dosage forms. U.S. Pat. No. 5,017,613 to Aubert et al. describes a process for preparing a composition containing valproic acid in combination with valproate sodium. A mixture of valproic acid and ethylcellulose is prepared and valproate sodium is added to the mixture to form drug granules in the absence of any binder or granulating solvent. Precipitated silica is added to the granules before the granules are compressed into tablets. U.S. Pat. Nos. 5,212,326 and 4,988,731 to Meade describe divalproex sodium and its preparation. Divalproex sodium is a stable 1:1 ionic oligomer in which valproic acid forms coordinate bonds with the sodium of the sodium valproate salt.

Sustained release forms of divalproex sodium, valproic acid and its salts and derivatives have been developed in an effort to minimize the gastrointestinal side effects associated with these compounds. For example, U.S. Pat. No. 5,807,574 to Cheskin et al. describes a controlled release dosage form containing divalproex sodium and a process for its preparation. The process involves melting divalproex sodium and mixing it with a molten wax to form a divalproex sodium-wax composite. The drug-wax mixture is formulated into a capsule. U.S. Pat. No. 5,169,642 to Brinker et al. describes a sustained release dosage form containing granules of divalproex sodium, valproic acid or amides or esters or salts thereof and a polymeric viscosity agent. The drug is coated with a sustained release composition comprising specified portions of ethylcellulose or a methacrylic methylester, a plasticizer, and a detactifying agent.

Enteric-coated dosage forms are typically produced by a film coating process, where a thin film layer of an acid-insoluble (enteric) polymer is applied to the surface of a pre-manufactured dosage form, such as a tablet, and to a lesser extent hard and soft capsules. The enteric coating is sprayed as an aqueous or organic solution or suspension of one or more enteric polymers onto tumbling or moving tablets or capsules, followed by drying at elevated temperatures. Enteric dosage forms made by this coating method can suffer from various process-related problems that affect the performance and/or appearance of the coating. For example, “orange peel” surface formation, also known as surface roughness or mottling, may result. More seriously, coat integrity failure may occur, such as cracking or flaking off of the enteric polymer coating.

U.S. Pat. No. 5,068,110 to Fawzi et al. describes various currently marketed delayed-release tablets and capsules, including the delayed-release divalproex sodium tablets manufactured by Abbott Laboratories (Depakote® ER). Fawzu states that the stability of the enteric coated capsules is increased by applying thicker layers of the enteric coating, alone or in combination with hydroxypropyl cellulose or hydroxymethylcellulose.

All coating processes present inherent problems, including possible uneven distribution of the coating ingredients, which can occur under multivariate coating processes. These problems are common to all enteric dosage forms. However, the problems faced during the coating of gelatin or polysaccharide capsules are even more critical due to the delicate and heat sensitive nature of the soft elastic capsule shell. Both hard and soft capsules can undergo thermally induced agglomeration and distortion of the capsule shell. Moreover, the smoothness and elasticity of the capsule surface makes it difficult to form an intact adhering enteric coating, without a subcoating step to improve the surface of the capsule for coating. Moreover, the enteric coatings cause the loss of the normally shiny and clear appearance of gelatin capsule shells, which is a major reason for the popularity and acceptance of gelatin capsules. WO 2004/030658 to Banner Pharmacaps, Inc. describes a process and resulting enteric capsule which avoids these problems with most drugs by incorporating the enteric polymer into the gelatin, rather than onto the gelatin.

It is therefore an object of the present invention to provide an enteric valproic acid soft gelatin capsule dosage form which does not suffer from the processing limitations and poor stability associated with traditional enteric coated dosage forms.

It is another object of the present invention to provide an enteric valproic acid soft gelatin capsule dosage form which minimizes the gastrointestinal side effects associated with valproic acid.

It is yet another object of the present invention to provide an enteric valproic acid soft gelatin capsule dosage form which is smaller, uses fewer ingredients, and is therefore easier to swallow, than conventional enteric valproic acid dosage forms.

It is still another object of the present invention to provide a method of making an enteric valproic acid soft gelatin capsule dosage form which is more economical than other methods.

### SUMMARY OF THE INVENTION

An enteric valproic acid soft gelatin capsule, in which the enteric polymer is a component of the capsule shell rather than a coating, has been developed. The fill material comprises valproic acid or divalproex sodium and, optionally, one or more pharmaceutically acceptable excipients such as corn oil. The capsule shell is prepared from a mass comprising a film-forming polymer, an acid insoluble polymer, an aqueous solvent, and optionally a plasticizer. Suitable film-forming polymers include gelatin. Suitable acid-insoluble polymers include acrylic-acid/methacrylic acid copolymers. The acid-insoluble polymer is present in an amount from about 8% to about 20% by weight of the wet gel mass. The weight ratio of acid-insoluble polymer to film-forming polymer is from about 25% to about 50%. The aqueous solvent is water or an aqueous solution of alkalis such as ammonia or diethylene amine or hydroalcoholic solutions of the same. Suitable plasticizers include glycerin and triethylcitrate.

The enteric soft gelatin capsule does not require an enteric coating and thus is not susceptible to the processing problems associated with enteric coated dosage forms. Enteric valproic acid soft gelatin capsules can be smaller in size and thus easier to swallow than currently available enteric coated tablets due to the presence of fewer ingredients, as well as smaller amounts of ingredients, in the capsule shell. In addition, the cost of manufacture due to the fewer processing steps and ingredients, is significantly less than with other methods.

### DETAILED DESCRIPTION OF THE INVENTION

I. Composition

A. Capsule Fill
1. Valproic Acid

Valproic acid, or 2-propylpentanoic acid, and its salts and derivatives are compounds which have been used to treat absence seizures, complex partial seizures, mania, migraine headache prophylaxis, and behavior dyscontrol. Valproic acid (available from Sifa Ltd., Shannon, Ireland; Interchem and Katwijk Chemie, the Netherlands; and Generichem) is an oily liquid at room temperature. Valproic acid is colorless and has a characteristic odor. It is slightly soluble in water (1.3 mg/mL) and very soluble in organic solvents. Valproic acid can be used neat or as a solution. The concentration of valproic acid in the fill material is from about 25% to about 100% by weight of the fill material. In the preferred embodiment, divalproex sodium is present in the fill at a concentration of about 40% by weight of the fill. Total dosage per capsule is typically 250 mg, although 125 mg and 500 mg sizes are also useful.

Divalproex sodium can also be used in the formulation of enteric soft gelatin capsules. Divalproex sodium is a 1:1 molar ratio oligomer of free valproic acid and sodium valproate. Divalproex sodium (available from SST Corp., New Jersey) is a white, crystalline powder, which is soluble in water and alcoholic solvents such as methanol and ethanol, as well as organic solvents such as cyclohexane.
2. Excipients

The capsule fill may be prepared using a pharmaceutically acceptable carrier composed of materials that are considered safe and effective and may be administered to an individual without causing undesirable biological side effects or unwanted interactions. The carrier consists of is all components present in the pharmaceutical formulation other than the active ingredient or ingredients. As generally used herein “carrier” includes, but is not limited to, plasticizers, crystallization inhibitors, wetting agents, bulk filling agents, solubilizers, bioavailability enhancers, solvents, pH-adjusting agents and combinations thereof.

Suitable excipients include one or more solubilizers such as soybean oil, rapeseed oil, safflower oil, corn oil, olive oil, castor oil, oleic acid, medium chain triglycerides, mono- and diglycerides (available from Abitec Corp., Columbus, Ohio, under the tradename Capmul®), medium chain triglyceride esters (available from Abitec Corp., Columbus, Ohio, under the tradename Captex®), medium chain partial triglycerides (available from Sasol under the tradename Imwitor®), corn oil-PEG 6 complex (available from Gattefosse S.A., Saint Priest, France under the tradename Labrasol®), propylene glycol monolaurate (lauraglycol), long chain partial glycerides (available from Gattefosse S.A., Saint Priest, France under the tradename Maisine®), sorbitan monooleate (available from ICI under the tradename Span®), polysorbates (available from ICI under the tradename Tween®), ethoxylated castor oil (cremophors), bees wax, hydrogenated soybean oil, partially hydrogenated soybean oil, and acetylated triglycerides. In a preferred embodiment, the solubilizer is corn oil.

B. Capsule Shell

The capsule shell is prepared from a gelatin mass comprising a film-forming polymer, an acid-insoluble polymer which is present in an amount making the capsule resistant to the acid within the stomach, an aqueous solvent, and optionally, one or more plasticizers and/or colorants. Other suitable shell additives including opacifiers, colorants, humectants, preservatives, flavorings, and buffering salts and acids. Enteric capsule shells and a method of making the capsule shell are described in WO 2004/030658 to Banner Pharmacaps, Inc.
1. Film-forming Polymers

Exemplary film-forming polymers can be of natural or synthetic origin. Natural film-forming polymers include gelatin and gelatin-like polymers. Other suitable natural film-forming polymers include shellac, alginates, pectin, and zeins. Synthetic film-forming polymers include hydroxypropyl methyl cellulose, methyl cellulose, hydroxypropyl methyl cellulose acetate succinate, hydroxypropyl methyl cellulose phthalate, cellulose acetate phthalate, and acrylates such as poly(meth)acrylate. The weight ratio of acid-insoluble polymer to film-forming polymer is from about 15% to about 50%. In one embodiment, the film forming polymer is gelatin.
2. Acid-insoluble Polymers

Exemplary acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers (available under the tradename EUDRAGIT® from Rohm America Inc., Piscataway, N.J. as a powder or a 30% aqueous dispersion; or under the tradename EASTACRYL®, from Eastman Chemical Co., Kingsport, Tenn., as a 30% dispersion). In one embodiment, the acid-insoluble polymer is EUDRAGIT® L100, which is a methacrylic acid/methacrylic acid methyl ester copolymer. The acid-insoluble polymer is present in an amount from about 8% to about 20% by weight of the wet gelatin mass. The weight ratio of acid-insoluble polymer to film-forming polymer is from about 15% to about 50%.
3. Aqueous Solvent

Exemplary aqueous solvents include water or aqueous solutions of alkalis such as ammonia, sodium hydroxide, potassium hydroxide, ethylene diamine, hydroxylamine, tri-ethanol amine, or hydroalcoholic solutions of the same. The alkali can be adjusted such that the final pH of the gelatin mass is less than or equal to 9.0, preferably less than or equal to 8.5, more preferably less than or equal to 8.0. In one embodiment, the alkali is a volatile alkali such as ammonia or ethylene diamine.
4. Plasticizers

Exemplary plasticizers include glycerol, glycerin, sorbitol, polyethylene glycol, citric acid, citric acid esters such as triethylcitrate, polyalcohols with 3-6 carbons and combinations thereof. The plasticizer to polymer (film forming polymer plus acid-insoluble polymer) ratio is from about 10% to about 50% of the polymer weight.

II. Method of Manufacture

A. Capsule Fill

Valproic acid or divalproex is dispensed into a suitable container and, optionally, mixed with a diluting vehicle such as corn oil. The fill is deaerated prior to encapsulation in a soft gelatin capsule.

B. Capsule Shell

A method of making the capsule shell is described in WO 2004/030658 to Banner Pharmacaps, Inc. The enteric gelatin mass can be manufactured by preparing an aqueous solution comprising a film-forming, water soluble polymer and an acid-insoluble polymer and mixing the solution with one or more appropriate plasticizers to form a gelatin mass. Alternatively, the enteric gelatin mass can be prepared by using a ready-made aqueous dispersion of the acid-insoluble polymer by adding alkaline materials such as ammonium, sodium, or potassium hydroxides or other alkalis that will cause the acid-insoluble polymer to dissolve. The plasticizer-wetted, film-forming polymer can then be mixed with the solution of the acid-insoluble polymer. The gelatin mass can also be prepared by dissolving the acid-insoluble polymer or polymers in the form of salts of the above-mentioned bases or alkalis directly in water and mixing the solution with the plasticizer-wetted, film-forming polymer. The gelatin mass is cast into films or ribbons using heat controlled drums or surfaces. The fill material is encapsulated in a soft gelatin capsule using a rotary die. The capsules are dried under controlled conditions of temperature and humidity. The final moisture content of the shell composition is from about 2% to about 10% by weight of the capsule shell, preferably from about 4% to about 8% by weight by weight of the capsule shell.

III. Method of Use

Enteric valproic acid soft gelatin capsules can be used to administer valproic acid or divalproex sodium in dose equivalents of 125 mg, 250 mg, and 500 mg.

### EXAMPLES

### Example 1

### Enteric Gelatin Mass

A gelatin mass was made according to the formula below.

| | |
|---|---|
| Gelatin | 28.00% |
| Eudragit ® L100 | 9.00% |
| Glycerin | 15.4% |
| Triethyl citrate | 0.90% |
| Ammonium hydroxide | 0.05% |
| Water | 46.65% |

The acid insoluble polymer (Eudragit® L 100) was dissolved in an aqueous alkali solution (water and ammonium hydroxide). The film-forming polymer (gelatin), and any plasticizers (glycerin), colorants, or other shell additives were added to the acid insoluble polymer solution and the mixture was cooked via a hot-melt process. The water content of the gelatin mass was adjusted accordingly. The gelatin mass was deaerated and dropped into a receiver. The dropped gelatin mass was held in the receivers at a temperature between 110 and 140° F. until encapsulation.

### Example 2

### Enteric Soft Capsules with Valproic Acid Fill

The capsules were prepared using a conventional rotary die process. The enteric gelatin mass from Example 1 was cast as a thin ribbon. The appropriate fill mass was pumped into each die cavity in order to provide the appropriate fill weight. After the die cavities were filled, the ribbon was sealed to form capsules of the desired shape and size. The capsule were dried initially in a tumble dryer and then dried on trays in a drying tunnel until the desired hardness was achieved. The dried capsules are inspected, sized, printed, polished and packaged.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

1. An enteric valproic acid soft gelatin capsule comprising:
(a) a fill material comprising an active agent consisting essentially of valproic acid or divalproex sodium; and
(b) a capsule shell comprising a gelatin mass, film-forming water soluble polymer, an acid-insoluble polymer, and an aqueous solvent.

2. The capsule of claim 1, wherein valproic acid is present in an amount from about 25% to about 100% by weight of the fill.

3. The capsule of claim 1, wherein the concentration of divalproex sodium is from about 25% to about 100% by weight of the fill.

4. The capsule of claim 1 wherein the capsule contains a dosage of valproic acid, divalproex sodium, or mixture thereof selected from the group consisting of 125 mg, 250 mg, and 500 mg.

5. The capsule of claim 1 wherein the shell comprises additional components selected from the group consisting of plasticizers, coloring agents, opacifiers, humectants, preservatives, flavorings, and buffering salts and acids.

6. The capsule of claim 1, wherein the fill material further comprises on or more pharmaceutically acceptable excipients.

7. The capsule of claim 6, wherein the one or more excipients is selected from the group consisting of crystallization inhibitors, wetting agents, bulk filling agents, solubilizers, bioavailability enhancers, solvents, pH-adjusting agents, dyes, preservatives, solvents, surfactants, and combinations thereof.

8. The capsule of claim 6 wherein the excipient is inert solubilizer selected from the group consisting of soybean oil, rapeseed oil, sunflower oil, corn oil, olive oil, castor oil, oleic acid, medium chain triglycerides, mono- and diglycerides, medium chain triglyceride esters, medium chain partial triglycerides, corn oil-PEG 6 complex, propylene glycol monolaurate, long chain partial glycerides, sorbitan monooleate, polysorbates, ethoxylated castor oil, bees wax, hydrogenated soybean oil, partially hydrogenated soybean oil, and acetylated triglycerides.

9. The capsule of claim 8 wherein the solubilizer is corn coil.

10. The capsule of claim 1 wherein the film-forming polymer is of natural origin.

11. The capsule of claim 10 wherein the film forming polymer is a natural film forming material selected from the group consisting of gelatin, shellac, alginates, pectin, and zeins.

12. The capsule of claim 11 wherein the natural film-forming polymer is gelatin.

13. The capsule of claim 1 wherein the film forming polymer is of synthetic origin.

14. The capsule of claim 13 wherein the film-forming polymer is selected from the group consisting of hydroxypropyl methyl cellulose, methyl cellulose, hydroxypropyl methyl cellulose acetate succinate, hydroxypropyl methyl cellulose phthalate, and cellulose acetate phthalate.

15. The capsule of claim 1 wherein the acid-insoluble polymer is selected from the group consisting of cellulose acetate phthalate, cellulose acetate butyrate, hydroxypropyl methyl cellulose phthalate, alginic acid salts such as sodium or potassium alginate, shellac, acrylic acid-methylacrylic acid copolymers.

16. The capsule of claim 15 wherein the acid-insoluble polymer is an acrylic acid-methacrylic acid copolymer.

17. The capsule of claim 1 wherein the acid-insoluble polymer is present in an amount from about 8 to about 20% by weight of the wet gelatin mass.

18. The capsule of claim 17 wherein the acid-insoluble polymer is present in an amount of about 12% by weight of the wet gelatin mass.

19. The capsule of claim 1 wherein the weight ratio of the acid-insoluble polymer to film-forming polymer is from about 15% to about 50%.

20. The capsule of claim 1 wherein the aqueous solvent is water

21. The capsule of claim 1 wherein the aqueous solvent an aqueous solution of an alkali selected from the group consisting of ammonia, sodium hydroxide, potassium hydroxide, hydroxyl amine, triethanol amine, and ethylene diamine.

22. The capsule of claim 21 wherein the aqueous solvent is present in an amount sufficient to give a final pH of the gelatin mass of less than or equal to 9.0.

23. The capsule of claim 21 wherein the aqueous solvent is present in an amount sufficient to give a final pH of the gelatin mass of less than or equal to 8.5.

24. The capsule of claim 21 wherein the aqueous solvent is present in an amount sufficient to give a final pH of the gelatin mass of less than or equal to 8.0.

25. The capsule of claim 1 wherein the plasticizer to polymer ratio is from about 10% to about 50% of the polymer weight.

26. The capsule of claim 1 wherein the final moisture content of the capsule is from about 2% to about 10% by weight of the capsule.

27. The capsule of claim 26 wherein the final moisture content of the capsule is from about 4% to about 8% by weight of the capsule.

28. The capsule of claim 3, wherein the concentration of divalproex sodium is about 40% by weight of the formulation.

29. A method for administering valproic acid, the method comprising administering to a patient in need thereof an entire enteric valproic acid soft gelatin capsule comprising:
(a) a fill material comprising an active agent consisting essentially of valproic acid or divalproex sodium; and
(b) a capsule shell comprising a gelatin mass, film-forming water soluble polymer, an acid-insoluble polymer, and an aqueous solvent.

30. The method of claim 28, wherein the enteric valproic acid soft gelatin capsule is administered to a patient suffering from a disorder selected from the group consisting of absence, seizures, complex partial seizures, mania, migraine headache prophylaxis, and behavior dyscontrol.

31. The method of claim 28, wherein the capsule contains a dosage of valproic acid, divalproex sodium, or mixture thereof selected from the group consisting of 125 mg, 250 mg, and 500 mg.
